(19) United States
(12) Patent Application Publication
(10) Pub. No.: US 2024/0115315 A1
(43) Pub. Date: Apr. 11, 2024

(54) ABLATION INSTRUMENT

(71) Applicant: Erbe Elektromedizin GmbH, Tuebingen (DE)

(72) Inventors: Hanna ANDEL, Moessingen (DE); Felix KARCHER, Tuebingen (DE); Marcus ADLER, Burladingen (DE)

(21) Appl. No.: 18/481,541

(22) Filed: Oct. 5, 2023

(30) Foreign Application Priority Data
Oct. 7, 2022 (EP) 22200312.1

(51) Int. Cl. A61B 18/14

(52) U.S. Cl. CPC A61B 18/1492; A61B 2018/00577

(57) ABSTRACT

An ablation instrument includes one or more electrodes that are configured as helical springs having ends for electrical connection that are configured as axial extensions. The electrodes are located on an inner hose serving as support, wherein the extensions are located at a smaller distance to the longitudinal axis than the windings of the electrode. Between the extension and the winding, a winding section is configured along which the distance toward the longitudinal center axis decreases from the radius of the winding to a lower value, so that the extension is located inward relative to the windings, i.e., closer to the center axis. For receipt of this extension and the associated electrical connection, the inner hose includes a trough.

### CROSS REFERENCE TO RELATED APPLICATION

This application claims the benefit of European Patent Application No. 22200312.1, filed Oct. 7, 2022, the contents of which are incorporated herein by reference as if fully rewritten herein.

### TECHNICAL FIELD

The invention refers to an ablation instrument, particularly a flexible ablation instrument, particularly for coagulation of tissue during so-called radio frequency ablation.

### BACKGROUND

Ablation probes of this type are in general known, as obvious from EP 0 754 075 B1, WO 94/11059, EP 2 768 563 B1 or EP 1 181 896 A1. The use of such instruments during coagulation of lung tumors is apparent from EP 3 763 314 A1.

During radio frequency ablation the ablation probe is located directly in the tissue structure to be treated with its active distal end. For example, the tissue is lung, liver or another tissue in which tissue is located that requires treatment, e.g., tumors. At the distal end of the probe one or more electrodes are provided that are supplied with high frequency alternating current. The latter creates a thermal necrosis in the tissue. To avoid undesirable heating, the electrodes can be actively cooled. EP 2 309 941 B1 and EP 3 769 706 A1 propose for this purpose to provide the distal end of an ablation probe with an internal cooling.

Particularly during treatment of lung tissue, it is necessary to guide the ablation instrument through the bronchial tree with bronchial tubes becoming smaller up to the tissue requiring treatment. In order to thereby reach practically any area of the lung parenchyma, on one hand a very small diameter of the ablation instrument and on the other hand a flexibility thereof being as high as possible is desired, so that bending radii being as small as possible, in the ideal case of less than 15 mm and high angles, in the ideal case of more than 145°, can be achieved. On the other hand, the probe shall be sufficiently stiff in order to allow piercing into the respective tissue to be treated. In addition, sufficient medical strength is required to be able to withstand stresses of the use, particularly occurring tensile forces and pressure created by cooling in the instrument. Similar problems can also occur in other tissues that can only be reached by endoscopy.

### SUMMARY

Therefrom one underlying object of the present invention is derived to provide a concept in which an ablation probe can be provided, which is concurrently extremely flexible and mechanically stable.

This object is solved by means of an ablation probe as described herein.

The ablation instrument according to the invention comprises a flexible hose arrangement on which at least one first electrode is arranged. In a preferred case at least one additional electrode can be arranged on the hose arrangement. The electrode is configured in the type of a helical spring and thus comprises multiple windings that wind around a center axis with constant radius and are arranged following a helical line. The center axis defines a longitudinal direction corresponding to the longitudinal direction of the flexible hose arrangement. At least a terminal winding of the electrode is connected with an extension that serves for electrical connection and projects from the remaining electrode. The extension is thereby orientated parallel or in an acute angle relative to the longitudinal direction, i.e. the center axis.

Between the extension extending substantially axially and the winding connected therewith a winding section is arranged, which on one hand is continuously orientated in circumferential direction, however, thereby comprises a radius (distance to the center axis) decreasing toward the extension. Due to this shape, the electrical connection of the electrode can be realized below an insulating body, which does not project beyond the radius of the electrode. Likewise the electrical connection can be located below an adjacent electrode or also proximal relative thereto. In doing so, it is achieved that no part of the instrument projects beyond the outer contour (being cylindrical in the stretched position) of the ablation instrument and indeed also not, if the instrument is highly angled with small bending radius. The electrodes do not form stiff locations of the instrument, i.e. they do not substantially reduce its flexibility. During bending of the instrument in the electrode area no parts of the electrodes or other parts of the instrument are urged outwardly, particularly also not the terminal winding.

The flexible hose arrangement preferably comprises an inner hose and a coating arranged thereon. For example, the inner hose can consist of the plastic PEEK. It supports one or more electrodes in an area where the coating is removed, wherein the inner hose does not provide any interruption for the current supply to the electrodes. Rather it is configured in an interruption-free manner (i.e. without any openings passing through the wall). The current supply line of each electrode can be arranged between the coating and the inner hose. For this purpose, a gap can be provided between the coating and the inner hose. For example, the coating can be made from the plastic PA. In addition, the plastic can be provided with a slip agent. Thereby a relative longitudinal movement between the inner hose and the coating can be allowed. In addition, the slip agent can facilitate the insertion of the instrument in the working channel of an endoscope.

The inner hose can comprise a region in which its cross-section deviates from the otherwise circular cross-section for location of the extension of the electrode as well as connection means which connect the extension with an electrical line. For this purpose, the inner hose can comprise a depression at least at one position, wherein the depression is orientated radially inwardly, e.g., forming a trough extending in a longitudinal direction. It can be depressed in the respective hose region by means of an embossing processing.

If two electrodes are present with longitudinal distance, an insulator can be arranged in between. Preferably, it comprises at least at one face side, preferably at both face sides a face following a helical line. In doing so, it is possible that the respective last winding of the helically shaped electrode abuts against the face side of the insulator without gap so that a homogeneous and edge-free transition between the electrode and the insulator is guaranteed. This results in an optimum insertion behavior of the instrument in a respective access providing system, such as an endoscope or the like. The instrument can be simply inserted into the working channel and can slide therein without getting caught anywhere.

In addition, the insulator can have a cavity for locating the extension of the electrode therein, so that the extension originating from the winding section can project into the trough formed in the inner hose without a sharp bent. Thereby, in addition, the bending resistance of the instrument is decreased that could be otherwise increased due to the extension and the electrical connection to a line, e.g. formed by means of a crimping barrel. The crimping barrel can be arranged adjoining the electrode or also with larger axial distance proximal thereto. Due to the cavity formed in the insulator, a minor radial movability of the extension and the winding section can be achieved, which is beneficial for the flexibility of the instrument.

In addition, the insulating body can comprise an additional cavity serving as adhesive reservoir. The adhesive can serve for securing the insulator and/or the electrode on the inner hose.

Further details of advantageous embodiments of the invention are subject of dependent claims, the drawing or the description.

### BRIEF DESCRIPTION OF THE DRAWINGS

The drawings show:

FIG. 1 an ablation instrument according to the invention that is connected to a schematically illustrated supply apparatus in a perspective overview illustration,

FIG. 2 the distal end of the ablation instrument in a longitudinally cut and schematic illustration not to scale,

FIG. 3 a cross-section of the instrument according to FIG. 2 cut along the line III-III,

FIG. 4 a cross-section of the instrument according to FIG. 2 cut along the line IV-IV,

FIG. 5 a front view of the electrode of the instrument according to FIGS. 1 and 2 in an illustration not to scale,

FIG. 6 a sectional view of the instrument according to FIG. 1 having two electrodes and an insulating body arranged therebetween,

FIG. 7 an electrical connection between the extension of the electrode and an electrical line formed by a crimping barrel.

### DETAILED DESCRIPTION

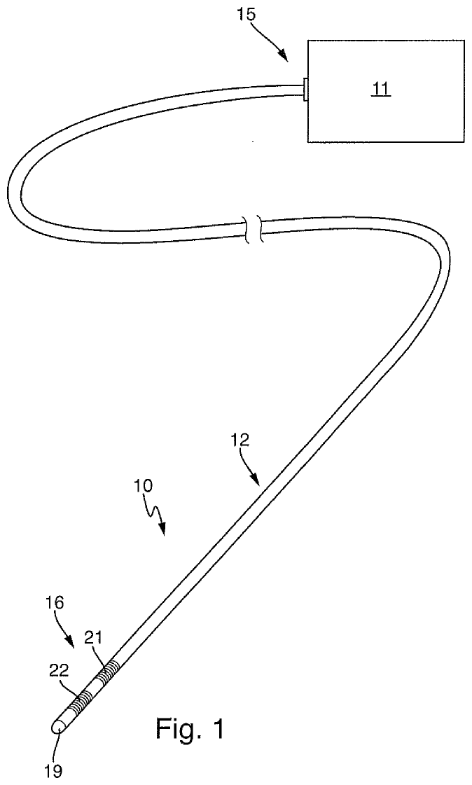

In FIG. 1 an instrument 10 is illustrated in form of a radio ablation instrument that is particularly provided and suitable for treatment of tissue in the lung, in general however, also for treatment of other tissue types. The instrument 10 is typically inserted through an endoscope in the bronchial tree of the lung in order to be subsequently precisely pierced there into tissue needing treatment. A respective endoscope as well as a patient to be treated are not illustrated in FIG. 1. The instrument 10 is connected to a supplying apparatus 11 or apparatus system that is only schematically indicated in FIG. 1. It serves for supply of instrument with coolant fluid and with electrical current for treatment of tissue.

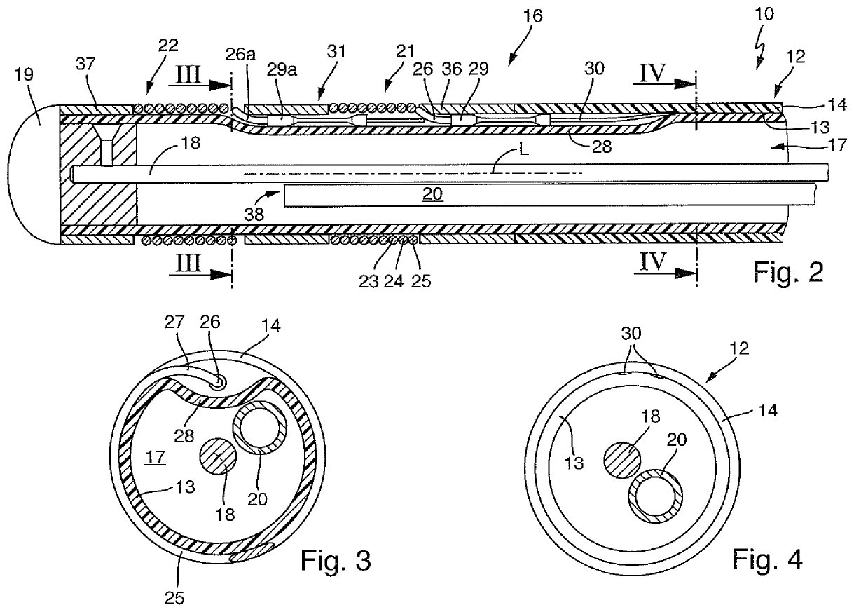

The instrument 10 comprises a hose arrangement 12, the configuration of which is particularly apparent from FIGS. 2-4. An inner hose 13 and a coating 14 arranged thereon are part of the hose arrangement 12. The inner hose 13 and the coating 14 are respectively made of plastic, preferably different plastics. The inner hose is preferably made from a plastically deformable plastic, e.g. PEEK, in which structures can be formed by means of embossing, such as grooves, depressions or the like. The coating is preferably made from a slidable plastic, e.g. PA. In addition, it can be provided with a sliding agent.

A lumen 17 extends through the inner hose from its proximal end 15 (FIG. 1) up to its distal end 16. A traction wire 18 extends through lumen 17, the distal end of which is connected to a closure piece 19 that closes lumen 17 on one end. The traction wire 18 can be connected to the closure piece 19 by substance bond, particularly by adhesive bond or by welding.

The closure piece 19 can have a head rounded distally and a shank that extends into the inner hose 13. The closure piece 19 can be made of plastic, ceramic or metal and forms the distal end of instrument 10. The closure piece 19 can also consist of multiple parts, e.g. an insulating body having one or more metal parts arranged therein that could serve as electrodes. By means of this electrode that can be potentially supplied with current via traction wire 18, the piercing of the instrument into the tissue is facilitated. Alternatively, the closure piece can be configured as massive metal part that is provided with an insulating coating on the outside. Uncovered locations on the distal end can serve as electrodes and, if they are supplied with current, e.g. via traction wire 18, the piercing of the ablation instrument into the tissue is facilitated.

The traction wire can be made of a metal having tensile strength, e.g. nitinol. It serves for transmission of tensile forces in the instrument 10 in order to be able to reliably retract it out of the tissue. The traction wire 18 can concurrently serve as electrical line, if the cover piece 19 shall in addition provide an electrical function as electrode. In addition, the preferably spring stiff traction wire 18, e.g. made of nitinol, supports the dimensional stability of the ablation instrument 10. Particularly after bending of the ablation instrument with small bending radii, the ablation instrument is does not remain deformed, but automatically resumes its stretched shape.

Inside lumen 17, in addition a supply hose 20 is arranged, which can extend from the proximal end 15 up to and into the distal end 16 and serves to supply the distal end 16 of instrument 10 with cooling fluid. The supply hose can also comprise one or more openings, via which the cooling fluid can be precisely released in the distal end 16 of instrument 10. The cooling fluid can then be fed back via the lumen to the apparatus 11.

On the inner hose 13, preferably in a section of distal end 16 where the coating is removed, at least one first electrode 21 is arranged that serves for supplying current to the tissue to be treated. Optionally an additional electrode 22 can be provided on the inner hose 13 with distal distance to the first electrode, wherein the additional electrode 22 can be preferably configured identically compared with the first electrode 21. The following description of the first electrode 21 thus also applies correspondingly also for the electrode 22, even without specific mention. Additional such electrodes can be provided as necessary.

The electrode 21 is formed by an electrical conductor that is formed following a helical line. The electrode 21 can be formed by a spring elastic wire, a sleeve provided with a helical slit, e.g. made of stainless steel, or the like and can be provided with a coating as necessary, e.g. a silver or gold coating. For example, the electrode 21 can have the shape of a helical spring having multiple windings 23, 24, 25. For electrical connection of electrode 21 the last winding 25 thereof, which is preferably the proximal winding, transitions into an extension 26 that extends substantially in longitudinal direction of instrument 10. The longitudinal direction of instrument 10 is indicated in FIG. 2 by means of a chain dotted line L. This chain dotted line L is concurrently the center axis of a helical line, which the windings 23, 24, 25 follow. Concurrently, the longitudinal axis L marks the longitudinal direction of instrument 10 (from distal to proximal) and thus also the longitudinal direction of hose arrangement 12 and thus the inner hose 13 and the coating 14.

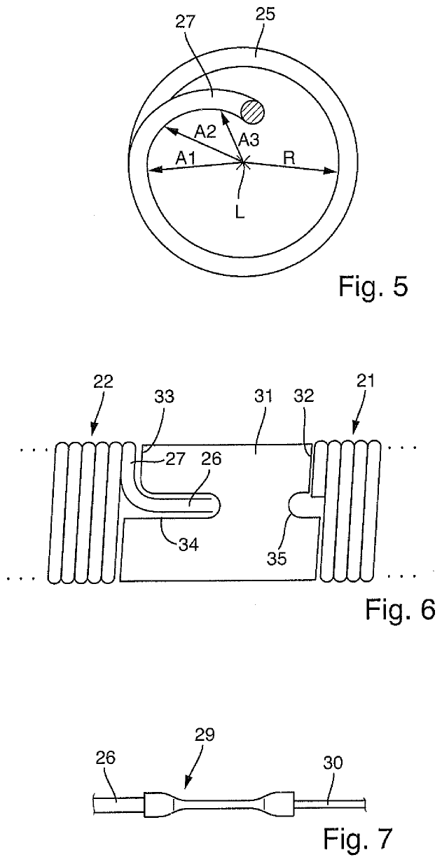

Between the extension 26 arranged preferably parallel to the longitudinal direction L (or in an acute angle thereto) and the winding 25 following the helical line, a winding section 27 is provided, which is in extension of winding 25 bent radially inwardly while extending in circumferential direction. In FIGS. 3 and 5 this is slightly excessively illustrated for sake of clarity. While windings 23, 24, 25 have a constant radius R, the distance decreases between the respective area of the winding section 27 and the center line L from a large distance A1 nearly corresponding to the radius R to a smaller distance A2 and to an even smaller distance A3. The winding section 27 extends along a part of the section of the electrode, preferably at least over 10 degrees or more.

At least in the range of extension 26 the inner hose 13 comprises a trough section deformed inwardly in which the electrical connection of electrode 21 is arranged. This connection is formed by suitable connection means, e.g. a crimp barrel 29. The crimp barrel 29 is an electrically conductive small tube in one end of which the extension 26 and in the other end of which an electrical line 30 is inserted, as shown in FIG. 7. The crimp barrel extends substantially in longitudinal direction L. It can have a stiffness that is larger than the stiffness of line 30 or extension 26.

The electrical line 30 extends from the crimp barrel 29 up to the proximal end 15 of instrument 10 and is there electrically connected with a generator that is not shown. Thereby line 30 is located in a gap between coating 14 and inner hose 13. If multiple electrodes 21, 22 are present, multiple of such lines 30 are provided correspondingly. For example, lines 30 can be configured as enameled wires, e.g. copper enameled wires, silver enameled wires or other suitable lines. The crimp barrel 29 can be insulated on its outer side by means of a shrinking hose, for example.

As shown in FIG. 2, crimp barrel 29a of second electrode 22 may extend inside trough 28 in a lying position below electrode 21. An electrical short circuit is effectively avoided by respective insulation of crimp sleeve 29a. The trough 28 can have a depth in radial direction that is longer than the outer diameter of crimp barrel 29a, so that crimp barrel 29a has a certain freedom of movement inside the trough. Alternatively, crimp barrel 29a can also be positioned proximally compared to the first electrode 21 inside trough 28, provided that extension 26a is configured with corresponding length. Crimping barrels 29, 29a as well as extensions 26, 26a are provided with a suitable insulation material, e.g. shrinking hose.

An insulator 31 can be provided between the two electrodes 21, 22, wherein insulator 31 can be made from a rigid or also flexible plastic, for example. The insulator can have faces 32, 33, as shown in FIG. 6, that have a shape adapted to the contour of the respective electrode 21, 22. The faces 32, 33 therefore follow the inclination of the windings of the two electrodes 21, 22. In doing so, the formation of open gaps of the steps between the electrode 21 and the insulator 31 is avoided, just as between insulator 31 and electrode 22. In addition, insulator 31 can have a cavity 34 that extends away from the face 33 and that is configured for locating the extension 26 therein. In doing so, it is avoided that the insulator presses the extension 26 radially inward and thus the part of the adjoining winding located opposite thereof radially outward. In addition, the flexibility of instrument 10 is improved in this area. The cavity 34 can provide a radial freedom of movement to extension 26 connected to the crimp barrel 29a, so that their stiffness is not transferred to the instrument.

An additional cavity 35 that originates from face 32 can be used as adhesive reservoir in order to fixate insulator 31 and/or electrode 21 on the inner hose 13.

The instrument can comprise a centering body 36 before first electrode 21 and coating 14. This centering body 36 can be configured on its face facing electrode 21 in a manner corresponding to face 33 of insulator 31 according to FIG. 6. On its side facing away from electrode 21 it can have a ring-shaped planar face adjoining the coating. The centering body 36 can be configured as slotted metal ring, which guarantees a defined ring gap between inner hose 13 and coating 14.

Similarly, an insulator 37 can be arranged between closure piece 19 and second electrode 22, whereby the insulator 37 abuts against closure piece 19 with at least one ring-shaped planar face. At its face facing electrode 22 it can be configured in a manner corresponding to the face 32 of insulator 31 according to FIG. 6.

The instrument 10 described so far operates as follows:

In medical use the instrument 10 is endoscopically inserted into the patient and the distal end 16 is pierced into the tissue to be treated so that electrode 21 is in contact with the tissue. If the instrument 10 comprises two electrodes 21, 22, it is pierced so that both electrodes 21, 22 are in contact with the tissue. The apparatus 11 then supplies current to the one or both electrodes 21, 22, which flows through the tissue to be ablated. Concurrently, cooling fluid is supplied into distal end 16 of instrument 10 via supply hose 20 in order to cool electrodes 21, 22. Used cooling fluid flows back to apparatus 11 through lumen 17.

The instrument 10 is configured extremely flexibly. During endoscopic insertion into the patient it is able to follow small bending radii. Thereby it can be bent particularly in the area of its distal end 16 also under deformation of electrodes 21, 22 without urging individual windings beyond the circular-shaped outer contour of instrument 10 thereby. The stiffness of crimp barrel 29a is largely decoupled from instrument 10, particularly from hose arrangement 12, i.e. in the context of the desired bending radii. In doing so, also tumors that are disadvantageously located or other tissue with need for treatment can be reached well. In addition, with the presented concept instruments having an outer diameter of less than 2 mm can be provided, whereby the instrument 10 can be inserted very deeply, also in narrow structures. In addition, inner hose 13 is configured without interruptions. The electrical lines 30 do not pass therethrough anywhere. Rather they are guided in a gap between inner hose 13 and coating 14. This increases safety against leakages, because no sealing locations are present in areas being under bending load. Also thereby instrument 10 is reliable against malfunction. The instrument 10 according to the invention comprises a high degree of patient safety.

An ablation instrument 10 according to the invention is characterized by at least one, preferably multiple electrodes 21, 22 that are configured in the type of helical springs and the respective ends of which provided for electrical connection are configured as axial extensions. The electrodes 21, 22 are located on an inner hose 13 serving as support, wherein the extensions are located at a smaller distance to the longitudinal axis than the windings of the electrode. Between the respective extension 26 and the respective winding 25 a winding section 27 is configured along which the distance toward the longitudinal center axis decreases from the radius R to a lower value, so that the extension 26 is located inward relative to the windings 23, 24, 25, i.e. closer to the center axis L. For receipt of this extension and the associated electrical connection, inner hose 13 comprises a trough 28. All in all, a reliable, flexible and concurrently robot configuration results.

### LIST OF REFERENCE SIGNS

10 instrument
11 apparatus
12 hose arrangement
13 inner hose
14 coating
15 proximal end
16 distal end
17 lumen
18 traction wire
19 closure piece
20 supply hose
21 first electrode
22 additional electrode
23-25 windings
L longitudinal direction
M center line/center axis
26 extension
27 winding section
28 trough section
29 crimp barrel
30 line
31 insulator
32, 33 faces
34, 35 cavities
36 centering body
37 insulator
38 exit opening

1. An ablation instrument (10) for RF-ablation, comprising:
a flexible hose arrangement (12) on which a first electrode (21) is arranged,
wherein the first electrode (21) comprises multiple windings (23, 24, 25) that wind around a center axis (M) and are arranged following a helical line, wherein the center axis (M) defines a longitudinal direction (L),
wherein the first electrode (21) comprises a terminal winding (25) that is connected to an extension (26) that extends away from the first electrode (21) parallel to the longitudinal direction (L) or at an acute angle thereto,
wherein a winding section (27) is arranged between the terminal winding (25) and the extension (26), and
wherein a distance (A) between the center axis (M) and the winding section (27) decreases from the terminal winding (25) towards the extension (26).

2. The ablation instrument according to claim 1, wherein the flexible hose arrangement (12) comprises at least one inner hose (13) and at least one coating (14) arranged thereon.

3. The ablation instrument according to claim 2, wherein the at least one inner hose (13) has a circular cross-section.

4. The ablation instrument according to claim 2, wherein the at least one inner hose (13) comprises a depression (28) oriented radially inwardly.

5. The ablation instrument according to claim 4, wherein the depression (28) is at least partly arranged below the first electrode (21).

6. The ablation instrument according to claim 1, further comprising at least one additional electrode (22) that is spaced apart from the first electrode (21) in the longitudinal direction (L).

7. The ablation instrument according to claim 6, wherein the at least one additional electrode (22) comprises multiple windings that wind around a second center axis and are arranged following a second helical line, wherein the second center axis defines a second longitudinal direction,
wherein the at least one additional electrode (22) comprises a second terminal winding that is connected to a second extension that extends away from the at least one additional electrode (22) parallel to the second longitudinal direction or at a second acute angle thereto,
wherein a second winding section is arranged between the second terminal winding and the second extension, and
wherein a second distance between the second center axis and the second winding section decreases from the second terminal winding towards the second extension.

8. The ablation instrument according to claim 6, wherein an insulator (31) is arranged between the first electrode (21) and the at least one additional electrode (22).

9. The ablation instrument according to claim 8, wherein the insulator (31) comprises at least one face (32, 33) following the helical line.

10. The ablation instrument according to claim 8, wherein the insulator (31) is configured in a hollow cylindrical manner and comprises a cavity (34) for location of the extension (26) therein.

11. The ablation instrument according to claim 8, wherein the insulator (31) comprises at least one adhesive reservoir.

12. The ablation instrument according to claim 11, wherein the at least one adhesive reservoir is formed by a cavity (34, 35) on at least one face of the insulator (31).

13. The ablation instrument according to claim 1, wherein the extension (26) is connected to an electrical line (30).

14. The ablation instrument according to claim 13, wherein the extension (26) and the electrical line (30) are connected to one another by a crimp barrel (29).

15. The ablation instrument according to claim 2, wherein the at least one inner hose (13) surrounds a lumen (17) inside of which a supply hose (20) is arranged having at least one exit opening (38).
